(19) United States
(12) Patent Application Publication
(10) Pub. No.: US 2004/0221810 A1
(43) Pub. Date: Nov. 11, 2004

(54) Process boat and shell for wafer processing

(76) Inventors: Ronald O. Miles, Menlo Park, CA (US); Ludwig L. Galambos, Menlo Park, CA (US); Janos J. Lazar, Redwood City, CA (US)

Correspondence Address: OKAMOTO & BENEDICTO, LLP, P.O. BOX 641330, SAN JOSE, CA, 95164, US

(21) Appl. No.: 10/865,101

(22) Filed: Jun. 9, 2004

Related U.S. Application Data
(63) Continuation-in-part of application No. 10/187,330, filed on Jun. 28, 2002.
(60) Provisional application No. 60/480,566, filed on Jun. 20, 2003.

(51) Int. Cl. A61M 36/14; A61K 51/00; C23C 16/00

(52) U.S. Cl. 118/715

(57) ABSTRACT

In one embodiment, an apparatus for wafer processing comprises a boat and a shell. The shell may be configured to receive and enclose the boat, which in turn may be configured to receive a plurality of wafers. The shell may include a plurality of slots to allow vapor to escape out of the shell and away from the wafers during a temperature ramp down. The apparatus may be employed in a variety of wafer processing applications including in processes for increasing the bulk conductivity of ferroelectric materials, for example.

### REFERENCE TO RELATED APPLICATIONS

This application is a continuation-in-part of U.S. application Ser. No.10/187,330, filed on Jun. 28, 2002, entitled “Method And Apparatus For Increasing Bulk Conductivity Of A Ferroelectric Material,” which is incorporated herein by reference in its entirety.

This application claims the benefit of U.S. Provisional Application No. 60/480,566, filed on Jun. 20, 2003, entitled “Process Boat And Shell For Wafer Processing,” which is incorporated herein by reference in its entirety.

This application is related to U.S. application Ser. No. ______, filed on the same day as this application by Ludwig L. Galambos, Joe McRae, and Ronald O. Miles, entitled “Method And Apparatus For Increasing Bulk Conductivity Of A Ferroelectric Material,” Attorney Docket No. 10021.001321 (P0314), which is incorporated herein by reference in its entirety.

### BACKGROUND OF THE INVENTION

1. Field of the Invention

The present invention relates generally to material processing, and more particularly, but not exclusively, to methods and apparatus for processing a ferroelectric material.

2. Description of the Background Art

Lithium tantalate (LiTaO₃) and lithium niobate (LiNbO₃) are widely used materials for fabricating nonlinear optical devices because of their relatively large electro-optic and nonlinear optical coefficients. These nonlinear optical devices include wavelength converters, amplifiers, tunable sources, dispersion compensators, and optical gated mixers, for example. Lithium tantalate and lithium niobate are also known as ferroelectric materials because their crystals exhibit spontaneous electric polarization.

Because lithium tantalate and lithium niobate materials have relatively low bulk conductivity, electric charge tends to build up in these materials. Charge may build up when the materials are heated or mechanically stressed. Because the charge may short and thereby cause a device to fail or become unreliable, device manufacturers have to take special (and typically costly) precautions to minimize charge build up or to dissipate the charge.

The bulk conductivity of a lithium niobate material may be increased by heating the lithium niobate material in an environment including a reducing gas. The reducing gas causes oxygen ions to escape from the surface of the lithium niobate material. The lithium niobate material is thus left with excess electrons, resulting in an increase in its bulk conductivity. The increased bulk conductivity prevents charge build up.

Although the just described technique may increase the bulk conductivity of a lithium niobate material under certain conditions, the technique is not particularly effective with lithium tantalate. A technique for increasing the bulk conductivity of a lithium tantalate material is desirable because lithium tantalate is more suitable than lithium niobate for some high-frequency surface acoustic wave (SAW) filter applications, for example.

### SUMMARY

In one embodiment, an apparatus for wafer processing comprises a boat and a shell. The shell may be configured to receive and enclose the boat, which in turn may be configured to receive a plurality of wafers. The shell may include a plurality of slots to allow vapor to escape out of the shell and away from the wafers during a temperature ramp down. The apparatus may be employed in a variety of wafer processing applications including in processes for increasing the bulk conductivity of ferroelectric materials, for example.

These and other features of the present invention will be readily apparent to persons of ordinary skill in the art upon reading the entirety of this disclosure, which includes the accompanying drawings and claims.

### DESCRIPTION OF THE DRAWINGS

FIG. 1 shows a schematic diagram of a container in accordance with an embodiment of the present invention.

FIG. 2 shows a schematic diagram of a housing in accordance with an embodiment of the present invention.

FIG. 3 shows a system for increasing the bulk conductivity of a ferroelectric material in accordance with an embodiment of the present invention.

FIG. 4 shows a flow diagram of a method of increasing the bulk conductivity of a ferroelectric material in accordance with an embodiment of the present invention.

FIG. 5 shows a schematic diagram of a wafer cage in accordance with an embodiment of the present invention.

FIG. 6 shows a manufacturing specification for a process boat in accordance with an embodiment of the present invention.

FIG. 7 shows a manufacturing specification for a shell in accordance with an embodiment of the present invention.

FIG. 8 shows a schematic diagram of a container in accordance with an embodiment of the present invention.

FIG. 9 shows a system for increasing the bulk conductivity of a ferroelectric material in accordance with an embodiment of the present invention.

FIG. 10 shows a flow diagram of a method of processing a ferroelectric material in accordance with an embodiment of the present invention.

The use of the same reference label in different drawings indicates the same or like components. Drawings are not necessarily to scale unless otherwise noted.

### DETAILED DESCRIPTION

In the present disclosure, numerous specific details are provided such as examples of apparatus, process parameters, process steps, and materials to provide a thorough understanding of embodiments of the invention. Persons of ordinary skill in the art will recognize, however, that the invention can be practiced without one or more of the specific details. In other instances, well-known details are not shown or described to avoid obscuring aspects of the invention.

Moreover, it should be understood that although embodiments of the present invention will be described in the context of lithium tantalate, the present invention is not so limited. Those of ordinary skill in the art can adapt the teachings of the present invention to increase the bulk conductivity of other ferroelectric materials such as lithium niobate, for example.

In accordance with an embodiment of the present invention, the bulk conductivity of a ferroelectric material may be increased by placing the material in an environment including metal vapor and heating the material to a temperature up to the Curie temperature of the material. Generally speaking, the Curie temperature of a ferroelectric material is the temperature above which the material loses its ferroelectric properties. By heating a single domain ferroelectric material to a temperature below its Curie temperature in the presence of a metal vapor with relatively high diffusivity, the ferroelectric domain state of the ferroelectric material is not appreciably degraded.

Preferably, the metal to be converted to vapor has relatively high diffusivity and has the potential to reduce the oxidation state of the ferroelectric material. The inventors believe that these properties will allow ions of the metal to diffuse a few microns into the surface of the ferroelectric material to fill lattice site vacancies, reducing the state of oxidation and thereby liberating electrons from the ferroelectric material and beginning a process of filling negative ion site vacancies throughout the bulk of the material. The electrons that fill these negative ion site vacancies are believed to be bound to point defect sites. These bound electrons, in general, will have a spectrum of energy levels that leave the ferroelectric material with a distinctive broad coloration. With the filling of lattice site vacancies and supplying neutralizing electrons to point defect sites , excess charge can be rapidly neutralized or conducted away perhaps as a polaron. When excess charge (electron) is introduced into the. lattice, it is energetically favorable for the electron to move as an entity within the polarization of the lattice. Such an entity, referred to as a “polaron”, results in increased electron mobility. Since the electron charge is screened by the lattice, polarons may move unobstructed by electrostatic forces along the lattice.

In one embodiment, the metal to be converted to vapor comprises zinc and the ferroelectric material comprises lithium tantalate in wafer form. Zinc vapor may be created by heating zinc to a temperature slightly below the Curie temperature of the lithium tantalate wafer. To obtain a vapor pressure that is high enough for efficient diffusion at a temperature below the Curie temperature, the metal and lithium tantalate wafer may be heated in a sealed container that has a predetermined volume. The inventors believe that heating a lithium tantalate wafer in zinc vapor causes zinc to diffuse into the surface of the lithium tantalate wafer and fill lithium site vacancies. It is believed that this results in the release of extra electrons according to equation 1:

Zn+VLi⁻═Zn⁺²Li+2e⁻ EQ. 1

It is believed that the extra electrons are trapped in negative ion site vacancies in the bulk of the lithium tantalate wafer. Increased electron mobility.in the bulk of the lithium tantalate wafer results when excess charge build up due to pyroelectric or piezoelectric effects are conducted away as polarons . That is, the inventors believe that the increased conductivity of the lithium tantalate wafer appears to be polaron in nature.

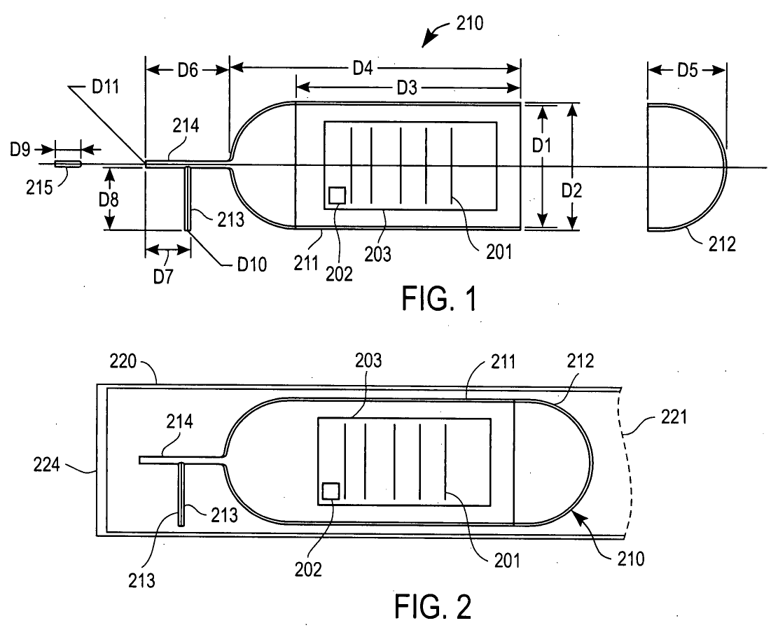

Referring now to FIG. 1, there is shown a schematic diagram of a container 210 in accordance with an embodiment of the present invention. Container 210 may be used to hold one or more wafers 201 to be processed and a metal 202 to be converted to vapor. Container 210 includes a body 211 and an end-cap 212. End-cap 212 may be welded onto body 211 using an oxygen-hydrogen torch, for example.

Body 211 includes a tube section 213 and a tube section 214. Container 210 may be sealed by capping tube sections 213 and 214, and welding end-cap 212 onto body 211. Tube section 214 may be capped by inserting a plug 215 into tube section 214 and welding the wall of plug 215 to that of tube section 214. Tube section 213 may be a sealed capillary tube. A vacuum pump may be coupled to tube section 214 to evacuate container 210. A sealed tube section 213 may be cracked open at the end of a process run to increase the pressure in container 210 (e.g., to bring the pressure in container 210 to atmospheric pressure).

Still referring to FIG. 1, one or more wafers 201 may be placed in a wafer cage 203, which may then be inserted into container 210. A metal 202 may be placed inside wafer cage 203 along with wafers 201. Wafer cage 203 may be a commercially available wafer cage such as of those available from LP Glass, Inc. of Santa Clara, Calif. Wafer cage 203 may be made of quartz, for example.

Table 1 shows the dimensions of a container 210 in one embodiment. It is to be noted that container 210 may be scaled to accommodate a different number of wafers.

TABLE 1
(REFER TO FIG. 1)

| Dimension | Value (mm) |
|---|---|
| D1 | Inside Diameter 120.00 |
| D2 | Outside Diameter 125.00 |
| D3 | 217.00 |
| D4 | 279.24 |
| D5 | 76.20 |
| D6 | 80.00 |
| D7 | 40.00 |
| D8 | 60.00 |
| D9 | 25.40 |
| D10 | Inside Diameter 4.00 |
| | Outside Diameter 6.00 |
| D11 | Inside Diameter 7.00 |
| | Outside Diameter 9.00 |

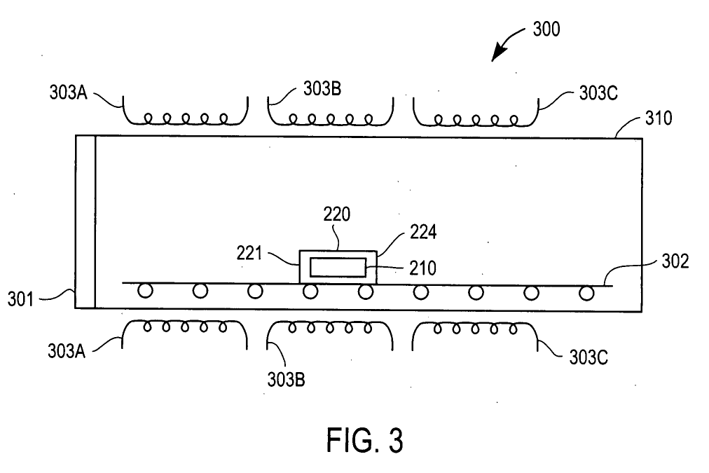

FIG. 2 shows a schematic diagram of a housing 220 in accordance with an embodiment of the present invention. Housing 220 may be a cylindrical container made of alumina. Container 210 may be inserted in housing 220, as shown in FIG. 2, and then heated in a process tube, as shown in FIG. 3. Housing 220 surrounds container 210 to allow for uniform heating of container 210. Additionally, housing 220 serves as a physical barrier to protect container 210 from breaking.

As shown in FIG. 2, housing 220 may have a closed-end 224 and an open-end 221. Container 210 is preferably placed inside housing 220 such that end-cap 212 is towards open-end 221. Open-end 221 allows for convenient removal of container 210 from housing 220. Open-end 221 also facilitates creation of a thermal gradient in container 210 during a temperature ramp down. The thermal gradient results in a cold spot in end-cap 212 that attracts precipitating metal vapor away from the wafers inside container 210. This minimizes the amount of precipitates that have to be removed from the surface of the wafers. This aspect of the present invention will be further described below.

FIG. 3 shows a system 300 for increasing the bulk conductivity of a ferroelectric material in accordance with an embodiment of the present invention. System 300 includes a process tube 310 containing housing 220. As mentioned, housing 220 houses container 210, which in turn holds metal 202 and wafers 201. Process tube 310 may be a commercially available furnace generally used in the semiconductor industry. Process tube 310 includes heaters 303 (i.e., 303A, 303B, 303C) for heating housing 220 and all components in it. Process tube 310 may be 72 inches long, and divided into three 24-inch heating zones with the middle heating zone being the “hot zone”. Process tube 310 may include a first heating zone heated by a heater 303A, a second heating zone heated by a heater 303B, and a third heating zone heated by a heater 303C. Process tube 310 also includes a cantilever 302 for moving housing 220, and a door 301 through which housing 220 enters and leaves the process tube. Housing 220 may be placed in the middle of process tube 310 with open-end 221 facing door 301.

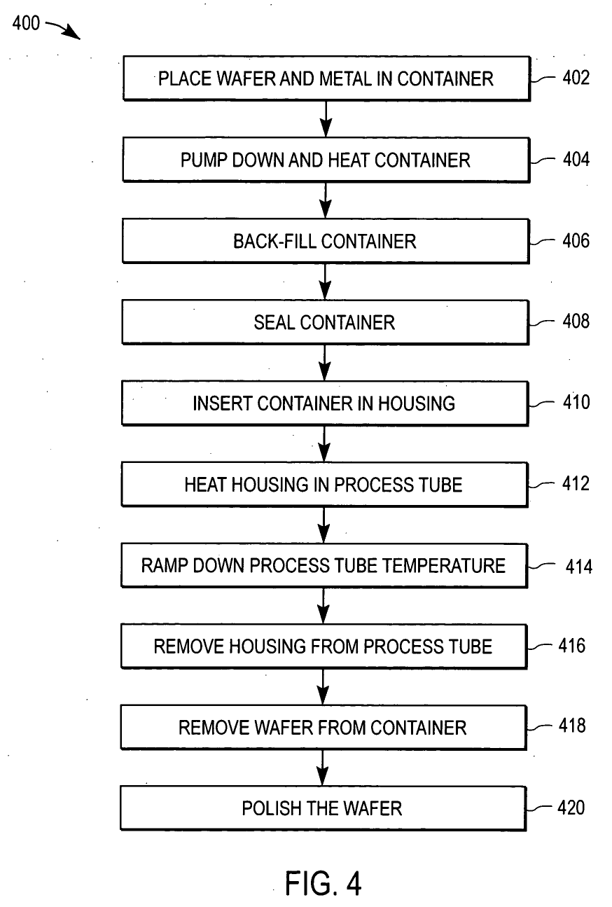

FIG. 4 shows a flow diagram of a method 400 for processing a ferroelectric material in accordance with an embodiment of the present invention. Method 400 will be described using container 210, housing 220, and system 300 as an example. It should be understood, however, that flow diagram 400, container 210, housing 220, and system 300 are provided herein for illustration purposes and are not limiting.

In step 402 of FIG. 4, metal 202 and one or more wafers 201 are placed in wafer cage 203. Wafer cage 203 is then placed inside container 210. In one embodiment, wafers 201 are 42 degree rotated-Y lithium tantalate wafers that are 100 mm in diameter, while metal 202 comprises zinc that is 99.999% pure. In one embodiment, five wafers 201 are placed in wafer cage 203 along with about 8 grams of zinc. The zinc may be in pellet form. Zinc pellets that are 99.999% pure are commercially available from Johnson Matthey, Inc. of Wayne, Pennsylvania. Note that the amount of zinc per wafer may be varied to suit specific applications.

In step 404, container 210 is pumped down to about 10⁻⁷ Torr and then heated to about 200° C. for about five hours. Step 404 may be performed by welding end-cap 212 onto body 211, capping tube section 213, coupling a vacuum pump to tube section 214, and heating container 210 with a heating tape wrapped around container 210. Step 404 helps remove oxygen sources, water, and other contaminants out of container 210 before metal 202 is melted.

In step 406, container 210 is back-filled so that the pressure in container 210 at slightly below Curie temperature is approximately 760 Torr. In one embodiment, container 210 is back-filled to about 190 Torr. This increases the pressure inside container 210, thus making it safer to heat container 210 to elevated temperatures for long periods of time. Container 210 may be back-filled with an inert gas such as Argon. Optionally, container 210 may be back-filled with forming gas comprising 95% nitrogen and 5% hydrogen. Note that the forming gas alone is not sufficient to reduce a lithium tantalate material so that its bulk conductivity is increased. However, in the present example, forming gas helps in trapping oxygen that may have remained in container 210 after step 404. Back-filling container 210 with forming gas may not be needed in applications where container 210 has been completely purged of contaminants. Container 210 may be back-filled by welding plug 215 to tube section 214, breaking the cap off tube section 213, and then flowing back-fill gas through tube section 213.

In step 408, container 210 is sealed. At this point, container 210 may be sealed by removing the source of the back-fill gas and capping tube section 213. (Note that end-cap 212 has already been welded onto body 211 and tube section 214 has already been capped in previous steps.)

In step 410, container 210 is inserted in housing 220.

In step 412, housing 220 is heated in process tube 310 at a temperature below the Curie temperature of wafers 201. Heating housing 200 at a temperature below the Curie temperature of wafers 201 melts metal 202 without substantially degrading the ferroelectric properties of wafers 201. Melting metal 202 results in metal vapor surrounding wafers 201. In this example, the metal vapor comprises zinc vapor and wafers 201 are of lithium tantalate. The interaction between zinc vapor and lithium tantalate that the inventors believe causes the bulk conductivity of wafers 201 to increase has been previously described above.

In one embodiment, housing 220 is heated in the middle of a process tube 310 that is 72 inches long. Also, as shown in FIG. 3, housing 220 may be placed in process tube 310 such that open-end 221 is facing door 301. Container 210 is preferably placed inside housing 220 such that end-cap 212 is towards open-end 221 (see FIG. 2).

In one embodiment, housing 220 is heated in process tube 310 at a ramp up rate of about 150° C./hour to a maximum temperature of about 595° C., for about 240 hours. Preferably, housing 220 is heated to a maximum temperature just a few degrees below the Curie temperature of wafers 201. Because the Curie temperature of wafers may vary depending on their manufacturer, the maximum heating temperature may have to be adjusted for specific wafers. The heating time of housing 220 in process tube 310 may also be adjusted to ensure adequate indiffusion of the metal vapor. Note that because method 400 is performed on bare wafers 201 (i.e., before devices are fabricated on wafers 201) the total process time of method 400 does not appreciably add to the amount of time needed to fabricate a device.

Continuing in step 414, the temperature inside process tube 310 is ramped down to prevent the just processed wafers 201 from being degraded by thermal shock. In one embodiment, the temperature inside process tube 310 is ramped down by setting its temperature set point to 400° C. Thereafter, cantilever 302 (see FIG. 3) may be programmed to move housing 220 towards door 301 at a rate of about 2 cm/minute for 3 minutes, with a 1.5 (one and a half) minute pause time between movements. That is, housing 220 may move at a rate of 3 cm/minute for 3 minutes, then pause for 1.5 minutes, then move at a rate of 3cm/minute for 3 minutes, then pause for 1.5 minutes, and so on for a total of 40 minutes until housing 220 reaches door 301.

As housing 220 is moved towards door 301, open-end 221 of housing 220 becomes cooler than closed-end 224. This results in a thermal gradient inside container 210, with end-cap 212 (which along with open-end 221 is facing door 301) becoming colder than the rest of container 210. The creation of a thermal gradient in container 210 may also be facilitated by adjusting the heaters of process tube 310 such that the temperature is lower towards door 301. The thermal gradient inside container 210 results in end-cap 212 becoming a cold spot that attracts precipitating metal vapor away from wafers 201.

In step 416, housing 220 is removed from process tube 310. Container 210 is then removed from housing 220.

In step 418, wafers 201 are removed from container 210. Step 418 may be performed by first cracking open tube section 213 (see FIG. 1) to slowly expose container 210 to atmosphere. Container 210 may also be back-filled with an inert gas. Thereafter, end-cap 212 may be cut away from body 211 using a diamond-blade saw, for example.

In step 420, wafers 201 are polished to remove precipitates from their surface and to expose their bulk. In one embodiment, both sides of a wafer 201 are polished by chemical-mechanical polishing to remove about 50 microns from each side.

In an experiment, five 42 degree rotated-Y lithium tantalate wafers that are 100mm in diameter, hereinafter referred to as “experimental wafers”, were processed in accordance with the just described method 400. The experimental wafers were placed in a container 210 along with 8 grams of zinc, and then heated in a process tube 310 to 595° C. for 240 hours. Thereafter, the temperature of the process tube 310 was ramped down and the experimental wafers were removed from the container 210. The experimental wafers were then polished on both sides and visually inspected. The experimental wafers looked homogenous and grayish in color. The bulk conductivity of the experimental wafers was then tested by placing them one at a time on a hot plate, raising the temperature of the hot plate from 80° C. to 120° C. at a rate of 3° C./min, and measuring the resulting electric field near the surface of the wafers. The electric field was measured using an electrometer from Keithley Instruments of Cleveland, Ohio under the model name Model 617. The experimental wafers did not produce any measurable electric field near their surface, indicating that their bulk conductivity has increased.

For comparison purposes, an unprocessed 42 degree rotated-Y lithium tantalate wafer that is 100 mm in diameter, referred to herein as a “control wafer”, was placed on a hot plate. The temperature of the hot plate was then increased from 80° C. to 120° C. at a rate of 3° C./min. Measuring the electric field near the surface of the control wafer indicated a 400V increase for every 20° C. change in temperature. This indicates that the bulk conductivity of the control wafer is relatively low.

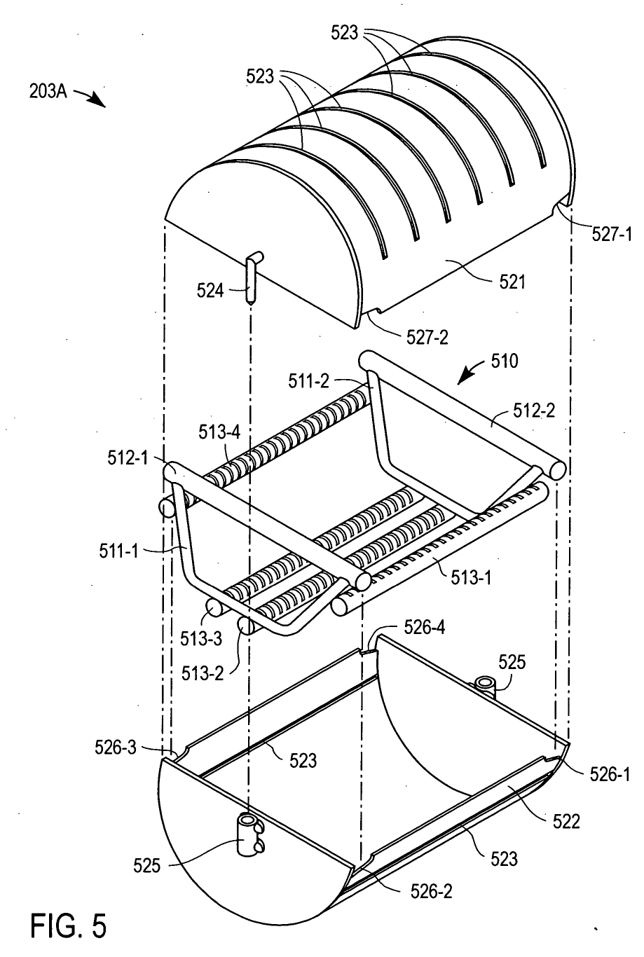

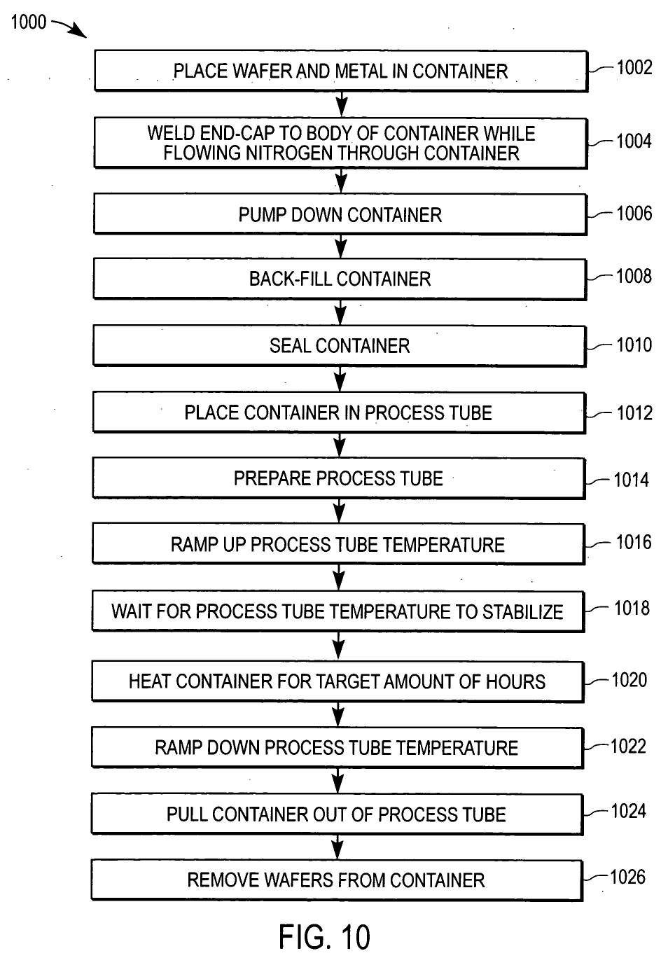

FIG. 5 shows a schematic diagram of a wafer cage 203A in accordance with an embodiment of the present invention. Wafer cage 203A is a specific implementation of wafer cage 203 shown in FIGS. 1 and 2. Wafer cage 203A may be employed in the process of method 400 or method 1000, which is later discussed in connection with FIG. 10. It should be understood, however, that wafer cage 203A is not so limited and may also be employed in other wafer processing applications. Furthermore, method 400 and method 1000 are not limited to the use of wafer cage 203, wafer cage 203A or the other apparatus disclosed herein. Methods 400 and 1000 may be performed using different wafer processing apparatus without detracting from the merits of the present invention.

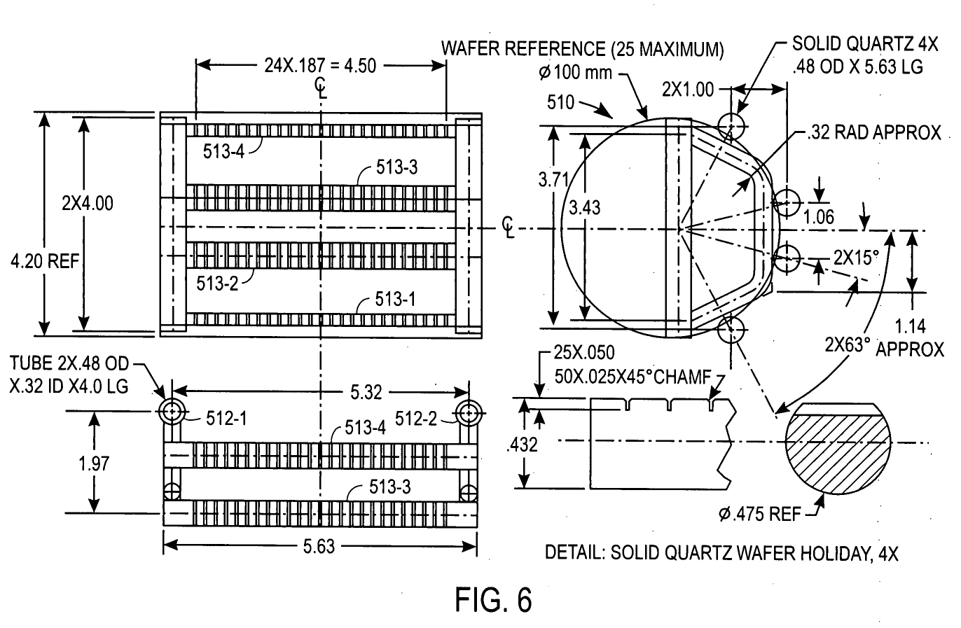

Wafer cage 203A comprises a process boat 510 and a shell comprising a top portion 521 and a bottom portion 522. Boat 510 comprises U-pieces 511 (i.e., 511-1, 511-2), bar pieces 512 (i.e., 512-1, 512-2), and rods 513 (i.e., 513-1, 513-2, 513-4). Rods 513 and U-pieces 511 form a structure for holding one or more wafers in boat 510. Rods 513 may have one or more notches (see FIG. 6), with each notch having a width that is wide enough to receive a single wafer. Wafer cage 203A may be made of quartz, for example. In that case, a laser may be employed to machine the notches on rods 513.

Bottom portion 522 of the shell includes clearances 526 (i.e., 526-1, 526-2, 526-3, 526-4). Each of clearances 526 forms a hole with a corresponding clearance 527 (i.e., 527-1, 527-2, 527-3, 527-4) of top portion 521. That is, when top portion 521 is placed over bottom portion 522, clearances 526-1 and 527-1 form a hole, clearances 526-2 and 527-2 form another hole, and so on. Clearances 527-3 and 527-4 of top portion 521 are not visible in FIG. 5.

Boat 510 may be placed and secured in bottom portion 522 by having bars 512 rest on clearances 526. For example, boat 510 may be placed in bottom portion 522 such that the ends of bar 512-1 settle on clearances 526-2 and 526-3, and the ends of bar 512-2 settle on clearances 526-1 and 526-4. Bars 512 may stick out of clearances 526 to allow an operator to readily pick-up boat 510 by the ends of bars 512. Top portion 521 goes over bottom portion 522 to enclose boat 510. Top portion 521 includes prongs 524 (one of which is not shown) that go into sockets 525 (one of which is not shown) of bottom portion 522 when the two portions are joined together to enclose boat 510.

When employed in a process where wafers are to be exposed to metal vapor (e.g., methods 400 and 1000), the shell advantageously helps contain metal vapor in the vicinity of the wafers during the main step of the process. During a temperature ramp down at the end of the process, however, metal vapor may turn into precipitates that may form on the surface of the wafers. The shell includes slots 523 to advantageously minimize the formation of precipitates on the wafers. During a temperature ramp down, the shell cools faster than the wafers enclosed therein, thereby attracting metal vapor to escape out of the shell and away from the wafers through slots 523. Slots 523 also prevent excessive pressure build-up within the shell.

FIG. 6 shows a manufacturing specification for a process boat in accordance with an embodiment of the present invention. FIG. 6 is for a specific implementation of boat 510. In the example of FIG. 6, the rods have 25 notches to accommodate 25 wafers. The boat of FIG. 6 may accommodate additional wafers by decreasing the pitch between notches. For example the pitch may be decreased to accommodate 50 wafers. The length of the rods may also be lengthened to accommodate more wafers. The dimensions in the example of FIG. 6 are in inches unless otherwise indicated.

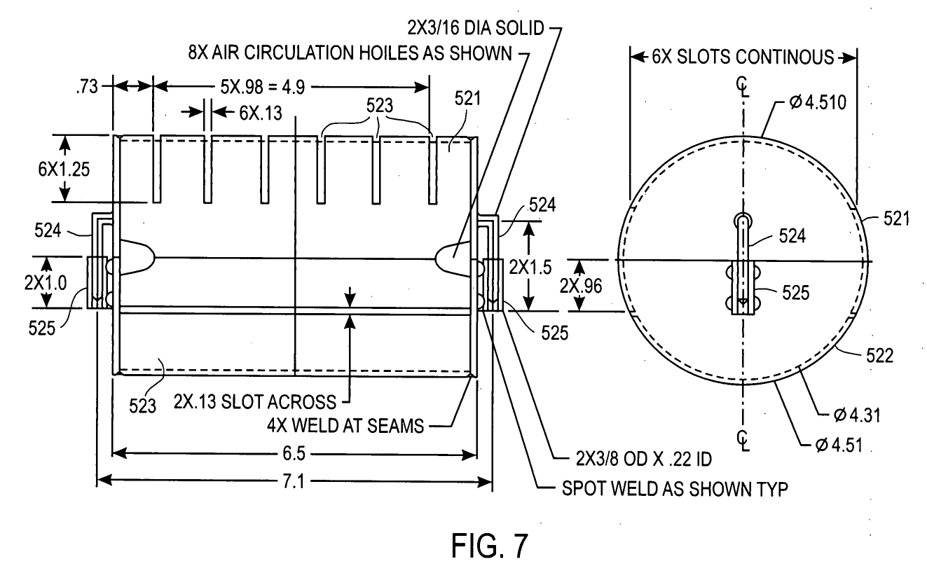

FIG. 7 shows a manufacturing specification for a shell in accordance with an embodiment of the present invention. FIG. 7 is for a specific implementation of the shell comprising top portion 521 and bottom portion 522 shown in FIG. 5. In the example of FIG. 7, the dimensions are in inches unless otherwise indicated.

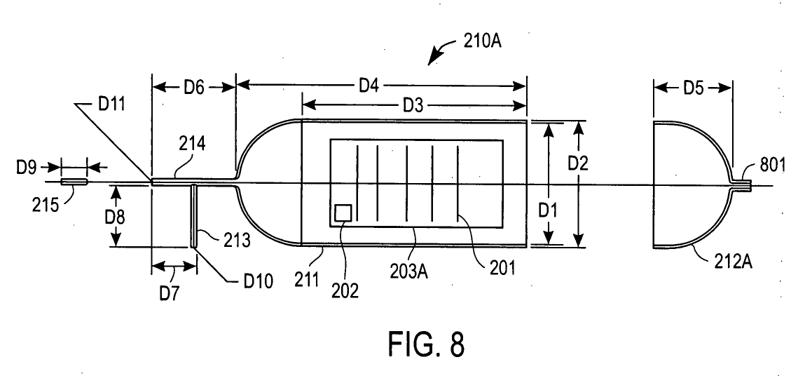

FIG. 8 shows a schematic diagram of a container 210A in accordance with an embodiment of the present invention. Container 210A is a specific implementation of container 210 shown in FIG. 1. Container 210A is the same as container 210 except for the addition of a nipple 801 in end-cap 212A. Reference labels common between FIGS. 1 and 8 indicate the same or similar components. Container 210A may be made of quartz, for example. Note that container 210A and other apparatus disclosed herein may be made of a material other than that disclosed without detracting from the merits of the present invention. Those of ordinary skill in the art will be able to select materials for the disclosed apparatus to meet the needs of specific applications.

As shown in FIG. 8, cage 203A may be used within container 210A.

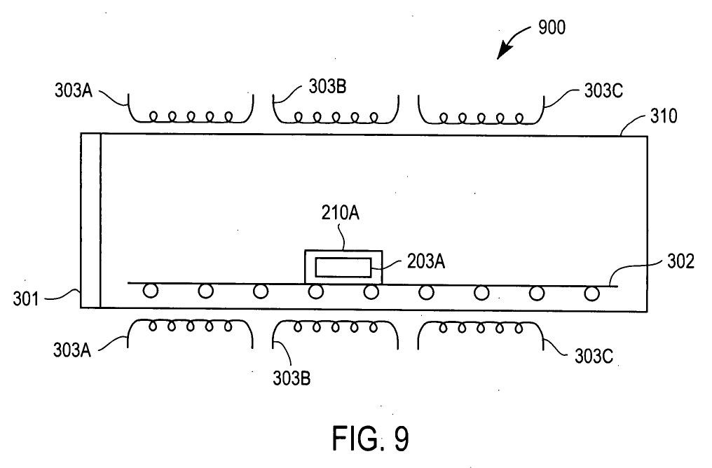

FIG. 9 shows a system 900 for increasing the bulk conductivity of a ferroelectric material in accordance with an embodiment of the present invention. System 900 is the same as system 300 shown in FIG. 3 except for the use of container 210A instead of container 210. Reference labels common between FIGS. 3 and 9 indicate the same or similar components. In one embodiment, system 900 does not include a housing enclosing container 210A. Wafers to be processed and a metal source (e.g., zinc pellets) may be placed in cage 203A, which in turn may be placed in container 210A.

FIG. 10 shows a flow diagram of a method 1000 for processing a ferroelectric material in accordance with an embodiment of the present invention. Method 1000 will be described using system 900 as an example, not a limitation.

In step 1002, a metal source and one or more wafers are placed in container 210A. Step 1002 may be performed by placing the wafers in boat 510, placing boat 510 and the metal source in bottom portion 522, covering bottom portion 522 with top portion 521, and then placing the resulting assembly (i.e., cage 203A) in body 211 of container 210A.

In step 1004, end-cap 212A of container 210A is welded onto body 211 to enclose cage 203A. Step 1004 may be performed by capping tube section 213 (see FIG. 8), leaving nipple 801 open, and flowing nitrogen gas into tube section 214 and out through nipple 801 during the welding process. The nitrogen gas serves as a drying agent that purges water vapor generated by the welding process out of container 210A.

In step 1006, container 210A is pumped down. Step 1006 may be performed by capping nipple 801, keeping tube section 213 capped, and coupling a pump to tube section 214. Container 210A does not have to be heated during step 1006. Pumping down container 210A helps remove oxygen sources, water, and other contaminants out of container 210A. Container 210A may be pumped down until the pressure within it has stabilized. In one embodiment, container 210A is pumped down for about 5 minutes.

In step 1008, container 210A is back-filled so that the pressure in container 210A at slightly below Curie temperature is approximately 760 Torr. Container 210A may be back-filled with an inert gas such as argon. Optionally, container 210A may also be back-filled with forming gas to trap oxygen that may have remained in container 210A after step 1006. Container 210A may be back-filled by welding plug 215 to tube section 214, breaking the cap off tube section 213, keeping nipple 801 capped, and then flowing back-fill gas through tube section 213.

In step 1010, container 210A is sealed. Container 210A may be sealed by removing the source of the back-fill gas, capping tube section 213, keeping tube section 214 capped, and keeping nipple 801 capped.

In step 1012, container 210A is placed in process tube 310 of system 900 (see FIG. 9). Container 210A may be placed in the middle of process tube 310, which in the example of FIG. 9 is the heating zone heated by heater 303B. Container 210A may be placed in process tube 310 at room temperature. Note that container 210A may be placed inside process tube 310 without a housing.

In step 1014, process tube 310 is prepared to run the process. Step 1014 may be performed by starting the flow of a nitrogen gas in the furnace. The nitrogen gas may be flowed continuously during the process run. at a flow rate of about 5 liters/min. The nitrogen gas helps preserve the integrity of components made of quartz, such as container 210A in this example.

In step 1016, the temperature inside process tube 310 is ramped up. In one embodiment, the temperature inside process tube 310 is ramped up at a rate of about 2.5° C./min to about 595° C. Depending on the particulars of the process tube employed, heaters 303A, 303B, and 303C may be configured such that the temperature in the middle section of the process tube where container 210A is placed is maintained at a target temperature (about 595° C. in this example) that is below a Curie temperature.

In step 1018, the temperature inside process tube 310 is allowed to stabilize. Step 1018 may be performed by waiting for about 25 minutes before proceeding to step 1020.

In step 1020, container 210A is heated for a target amount of time at a target temperature. The target temperature is preferably slightly below the Curie temperature of the wafers being processed, while the target amount of time may be varied to achieve a target wafer conductivity. For example, container 210A may be heated at a temperature of about 595° C. for about 25 hours or less. The inventors believe that heating time is proportionally related to bulk conductivity. That is, the longer the heating time, the higher the resulting bulk conductivity of the wafers. For example, a heating time of about 200 hours may result in the wafers having a bulk conductivity of about 10⁻¹⁰(Ωcm)⁻¹, while a heating time of about 25 hours may result in the wafers having a bulk conductivity of about 10⁻¹²(Ωcm)⁻¹. For comparison purposes, an unprocessed wafer may have a bulk conductivity of about 10⁻¹⁶(Ωcm)⁻¹. The heating time may thus be varied to meet the conductivity requirement of specific applications.

In step 1022, the temperature inside process tube 310 is ramped down to prevent the wafers from being degraded by thermal shock. In one embodiment, step 1022 is performed by ramping down the temperature in all heating zones of process tube 310 to about 530° C. at a rate of about 1.5° C./min.

In step 1024, container 210A is pulled out of process tube 310. In one embodiment, container 210A is pulled out of process tube 310 at a rate of about 3 cm/min using the following sequence:

a) pull container 210A out 15 cm, wait 1 minute;

b) pull container 210A out 15 cm, wait 1 minute;

c) pull container 210A out 10 cm, wait 1 minute and 10 seconds;

d) pull container 210A out 10 cm, wait 1 minute and 10 seconds;

e) pull container 210A out 10 cm, wait 1 minute and 10 seconds;

f) continue pulling out container 210A at 10 cm increments until 90 cm has been covered;

g) pull container 210A out the remaining distance to the opening of process tube 310 at a rate of 3 cm/min.

In step 1026, the wafers are removed from container 210A after container 210A has cooled down. The wafers may be wet etched or polished to remove precipitates that may have formed on their surface and to expose their bulk.

While specific embodiments of the present invention have been provided, it is to be understood that these embodiments are for illustration purposes and not limiting. Many additional embodiments will be apparent to persons of ordinary skill in the art reading this disclosure. Thus, the present invention is limited only by the following claims.

What is claimed is:

1. A wafer processing apparatus comprising:
a boat configured to receive a plurality of wafers; and
a shell configured to receive and enclose the boat and a metal source, the shell being configured to contain vapor of the metal source in a vicinity of the plurality of wafers during a main portion of a process, the shell including a plurality of slots configured to allow vapor of the metal source to escape out of the shell and away from the plurality of wafers during a temperature ramp down.

2. The apparatus of claim 1 wherein the shell comprises a top portion and a bottom portion that may be joined together to enclose the boat.

3. The apparatus of claim 1 wherein the vapor comprises zinc vapor.

4. The apparatus of claim 1 wherein the boat and the shell form a cage configured to be heated in a process tube.

5. The apparatus of claim 1 wherein the boat comprises:
a plurality rods having one or more notches and configured to receive an edge of a wafer in the plurality of wafers;
a plurality of U-pieces attached to the rods to form a structure for holding the plurality of wafers; and
a plurality of bar pieces configured to facilitate handling of the boat.

6. The apparatus of claim 5 wherein the shell comprises a top portion and a bottom portion that each has a clearance for accepting a bar piece in the plurality of bar pieces.

7. The apparatus of claim 6 wherein the top portion and the bottom portion have a mating socket and prong connection.

8. The apparatus of claim 1 wherein the plurality of wafers comprise lithium tantalate wafers.

9. The apparatus of claim 1 wherein the boat and the shell are made of quartz.

10. A wafer processing apparatus comprising:
boat means for receiving a plurality of wafers; and
shell means for receiving and enclosing the boat and a metal source, the shell means including slots to allow a vapor of a metal source to escape out of the shell during a temperature ramp down.

11. The wafer processing apparatus of claim 10 wherein the shell means comprises a top portion having the slots and a bottom portion supporting the boat.

12. The wafer processing apparatus of claim 10 wherein boat means and the shell means form a cage configured to be heated in a process tube.

13. The wafer processing apparatus of claim 10 wherein the plurality of wafers comprise lithium tantalate.

14. The wafer processing apparatus of claim 10 wherein the metal source comprises zinc.

15. The wafer processing apparatus of claim 10 wherein the boat means and the shell means are made of quartz.

16. An apparatus for wafer processing comprising:
a shell having a top portion and a bottom portion, the top portion including a plurality of slots to allow vapor to escape out of the boat substantially through the slots during a temperature ramp down; and
a boat configured to support a plurality of wafers, the boat being configured to be enclosed by the shell during processing of the wafers.

17. The apparatus of claim 16 wherein the boat further comprises:
a plurality rods having one or more notches and configured to receive an edge of a wafer in the plurality of wafers;
a plurality of U-pieces attached to the rods to form a structure for holding the plurality of wafers; and
a plurality of bar pieces configured to facilitate handling of the boat.

18. The apparatus of claim 16 wherein the boat and the shell form a cage configured to be heated in a process tube.

19. The apparatus of claim 16 wherein the plurality of wafers comprise lithium tantalate.

20. The apparatus of claim 16 wherein the shell and the boat comprise quartz.
